(19) United States
(12) Patent Application Publication
(10) Pub. No.: US 2022/0068504 A1
(43) Pub. Date: Mar. 3, 2022

(54) Automated Web-Based Second Opinion Brokerage System

(71) Applicant: USARAD HOLDINGS, INC., Fort Lauderdale, FL (US)

(72) Inventors: Michael Yuz, Oakland Park, FL (US); Alexander Krakopolsky, Oakland Park, FL (US); Nadya Kanevski, Oakland Park, FL (US)

(21) Appl. No.: 17/007,985

(22) Filed: Aug. 31, 2020

(51) Int. Cl. G16H 80/00; G06Q 10/10; G06Q 30/02; G16H 40/20

(52) U.S. Cl. CPC G16H 80/00; G16H 40/20; G06Q 30/0281; G06Q 10/1095

(57) ABSTRACT

A computer-implemented method includes the steps of receiving, at a processing device of a brokerage server, a request from a first remote computing device, the request being indicative of a request by a consumer for a second opinion from an expert, determining, at the processing device, a list of one or more experts who match the request, providing, from the processing device, the list of the one or more experts to the first remote computing device, and brokering, at the processing device, a consultation between the consumer and the expert.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, TABLE OR COMPUTER PROGRAM LISTING APPENDIX

Not Applicable

### BACKGROUND OF THE INVENTION

### 1. Field of the Invention

The present invention relates generally to systems and methods for providing second opinions. In particular, examples of the present invention are related to an automated web-based second opinion brokerage system for brokering a consultation between consumers with experts.

### 2. State of the Prior Art

A second opinion is an opinion received from a consultation with an expert other than the one a consumer originally consulted with in order to get an opinion from a differing point of view. The second opinion can be the opinion of a professional such as a medical doctor or of a lay expert such as an auto mechanic. There are many circumstances in which it is desirable for a consumer to have a second opinion such as when the consumer is a patient and needs a medical second opinion or when the consumer is in need of a legal second opinion.

The knowledge base of not only professionals but also of lay experts has dramatically expanded in virtually all fields with the continuing development of new technologies, equipment and increases in computing power and Internet speed. This development has made increasing amounts of information accessible to both professional and lay experts who in turn are accessible to consumers of all types by virtue of the computing devices used by such consumers. In addition, keeping up with technological and scientific advances presents challenges to all experts. Experts who are subspecialists in their fields have emerged in response to these developments and challenges. For example, a doctor who is also a radiologist may be a specialist, a bone radiologist a subspecialist, and a foot radiologist a super-specialist.

While many experts are competent in their field of expertise, skills, training and opinions vary. Experts, including physicians, have different personalities, different experiences and different training. Some experts are more conservative and others are more aggressive. Further, experts, being human beings, make mistakes. Studies have reported that mistake rates for experts can be 30% and higher. A second opinion from another expert can provide a consumer with insurance against these odds.

In medicine, the second opinion is often an important factor considered by a patient deciding on an ideal course of treatment. The opinion of a second physician can help avoid problems such as misdiagnosis, as well as give the patient peace of mind in knowing that a recommended procedure is the best course of treatment and is agreed upon by more than one professional. A second opinion also empowers a patient to make informed decisions in selecting their health care provider and where they receive their health care.

There are many reasons for a patient to seek a medical second opinion. Medical second opinions can be sought by a patient under circumstances including: 1) when a physician recommends surgery; 2) when a physician diagnoses the patient with a serious illness (such as cancer) that the patient does not believe he has; 3) when a physician recommends a treatment for the patient other than what the patient believes is necessary; 4) when the physician recommends elective surgery and a second opinion is required by an insurance policy; 5) when the patient believes that they have a condition that the physician fails to diagnose; and 6) when the physician recommends the second opinion.

Consumers with access to the Internet can seek a second opinion from medical professionals located miles or even continents away. For example, telemedicine services provide a means by which doctors can consult with each other while telehealth services enable healthcare providers to remotely connect with patients. These services include consultations using video conferencing, digital examination devices and clear voice conversations. Telemedicine products using the latest medical technology, including examination cameras, peripherals, scopes, remote vital sign monitors and fully-equipped telemedicine carts can be used to enhance second medical opinions. Patients can contact a physician or subspecialist directly. Or a primary care physician can facilitate a consultation with a physician or subspecialist on the patient's behalf by recommending a qualified physician or subspecialist.

The obvious value of the availability of a remote second opinion is that location is no longer a factor in the availability of a specific specialist's expertise. Telemedicine allows patients to connect with the ideal physician for their condition, regardless of geographic restrictions.

Depending upon the consumer's location, availability and ability to travel and the accessibility to highly specialized experts, the consumer may face barriers in getting a second opinion. In addition, most experts including medical doctors do not have access to telemedicine services and do not offer a convenient second opinion service.

### BRIEF SUMMARY OF THE INVENTION

In accordance with embodiments of the present disclosure, an automated web-based second opinion brokerage system for brokering a consultation between a consumer and an expert provides for the virtualization of second opinion brokerage services. Second opinion brokerage services including matching a consumer with an expert and providing communication, negotiation and consultation between the consumer and the expert are easily accessible via a transparent, secure, and on-demand web portal. In accordance with other embodiments of the present disclosure, the automated web-based second opinion brokerage system of the present disclosure can provide a consumer with information related to the subject matter of the second opinion being sought.

In accordance with embodiments of the present disclosure, the automated web-based second opinion brokerage system can include a web-based portal that enables registered consumers and experts to communicate with each other and with a brokerage server over a network. The communication can include sending a request from a consumer computing device to the brokerage server requesting a second opinion from an expert. As used herein, the term “computing device” can refer to mobile computing devices, such as mobile telephones, laptop computers, and tablet computers, or stationary computing devices. The request can include a description of the consumer and the nature of the second opinion he is seeking. The brokerage server can perform a search based on the request and send a list of experts that match the request to the consumer computing device. The consumer can select an expert from the list of experts and send the selection to the brokerage server. The brokerage server can serve as a broker between the consumer and the selected expert as the terms and conditions of a consultation between the consumer and the selected expert are negotiated.

In some embodiments of the present disclosure, the second opinion rendered during the consultation can be a lay opinion received from a consumer consultation with a lay expert. In other embodiments of the present disclosure, the second opinion can be an expert opinion received from a consumer consultation with a professional expert. For example, the second opinion can be a medical second opinion received from a patient consultation with a medical doctor. In yet other embodiments of the present disclosure, the second opinion can be an expert opinion received from a professional consultation with another professional. In some other embodiments, the second opinion can be an opinion generated by the brokerage server in response to a request from the consumer. For example, the brokerage server can employ artificial intelligence to build a database of opinions indexed to particular consumer requests.

### BRIEF DESCRIPTION OF THE DRAWING FIGURES

Non-limiting and non-exhaustive embodiments of the present invention are described with reference to the following figures, wherein like reference numerals refer to like parts throughout the various views unless otherwise specified.

FIGS. 1A-1G are schematics illustrating a consumer computing device and an expert computing device displaying a graphical user interface in communication with a brokerage server in accordance with some embodiments of the present disclosure;

FIG. 2 is a block diagram illustrating components of the computing devices of FIGS. 1A-1G;

FIG. 3 is block diagram illustrating components of the brokerage server of FIGS. 1A-1G; and

FIG. 4 is a flow chart illustrating an example method for brokering a second opinion consultation between a consumer and an expert in accordance with some embodiments of the present disclosure.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help improve the understanding of various embodiments of the present invention. Also, common but well-known elements that are useful or necessary in a commercially feasible embodiment are often not depicted in order to facilitate a less obstructed view of these various embodiments of the present invention.

### DETAILED DESCRIPTION OF A PREFERRED EMBODIMENT

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

Reference throughout this specification to “one embodiment”, “an embodiment”, “one example”, or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Embodiments in accordance with the present invention may be embodied as an apparatus, method, or computer program product. Accordingly, the present invention may take the form of an entirely hardware embodiment, an entirely software embodiment (including firmware, resident software, micro-code, etc.), or an embodiment combining software and hardware aspects that may all generally be referred to herein as a “module” or “system.” Furthermore, the present invention may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present invention may be written in any combination of one or more programming languages.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

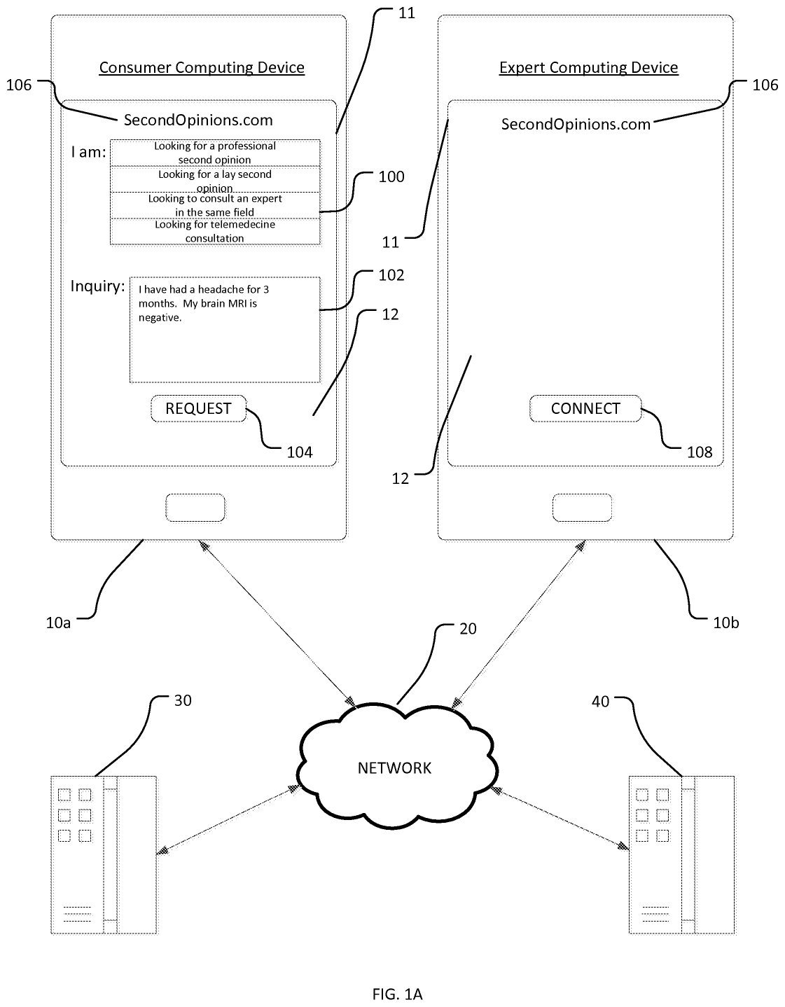

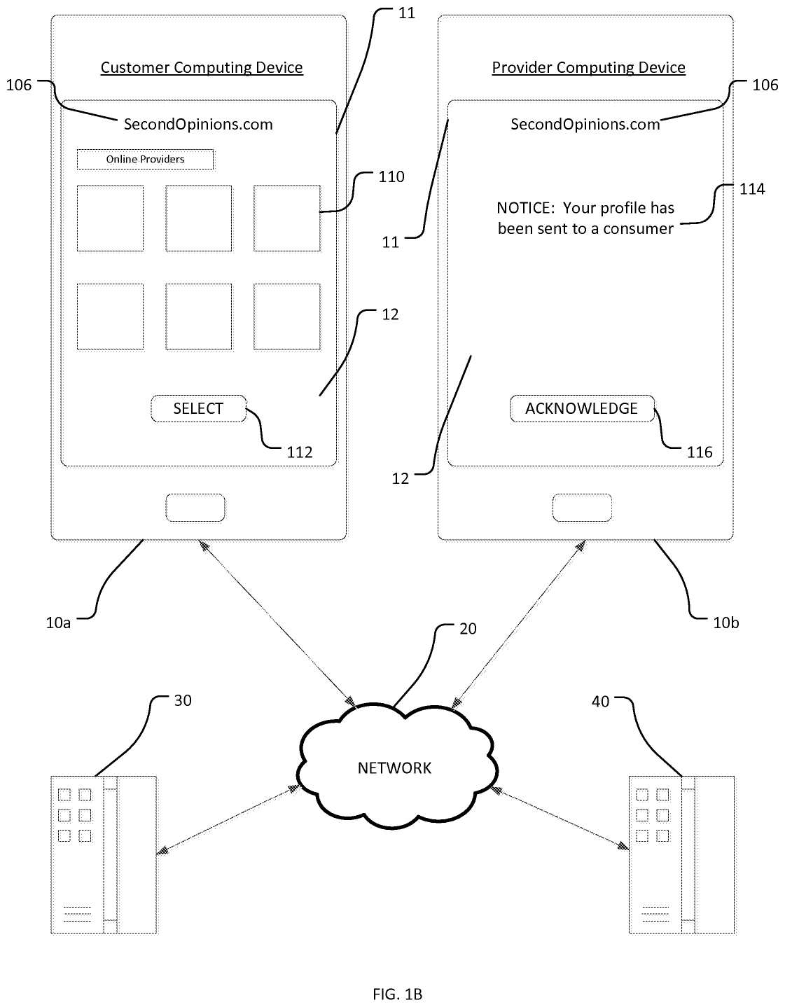

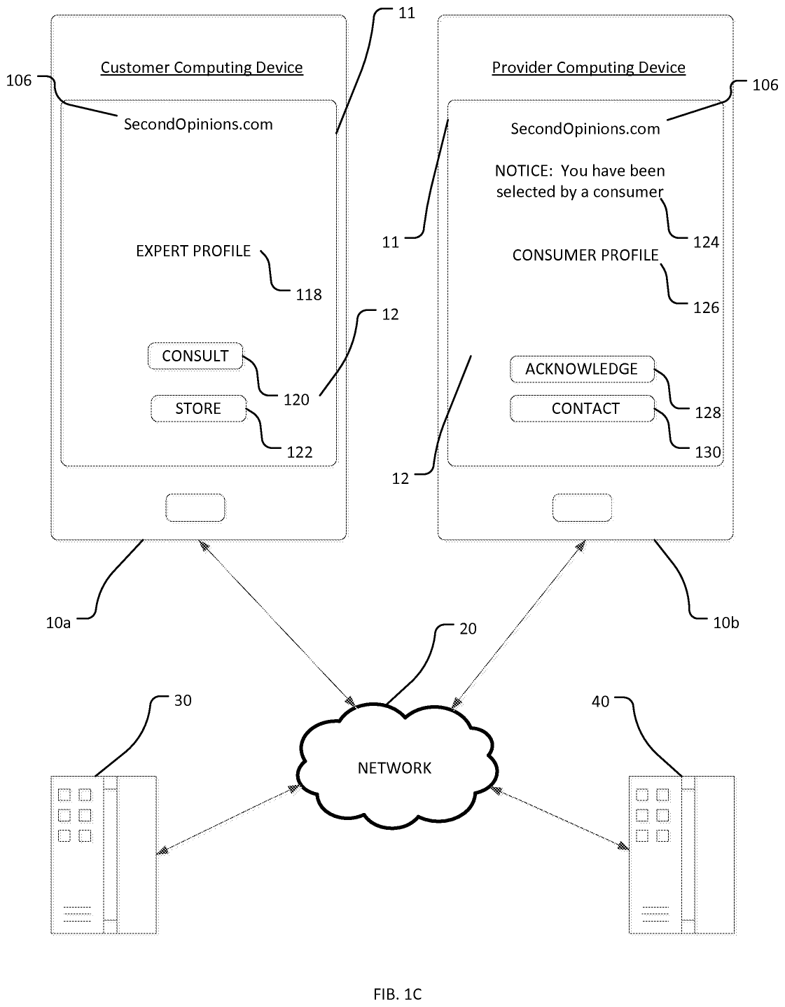

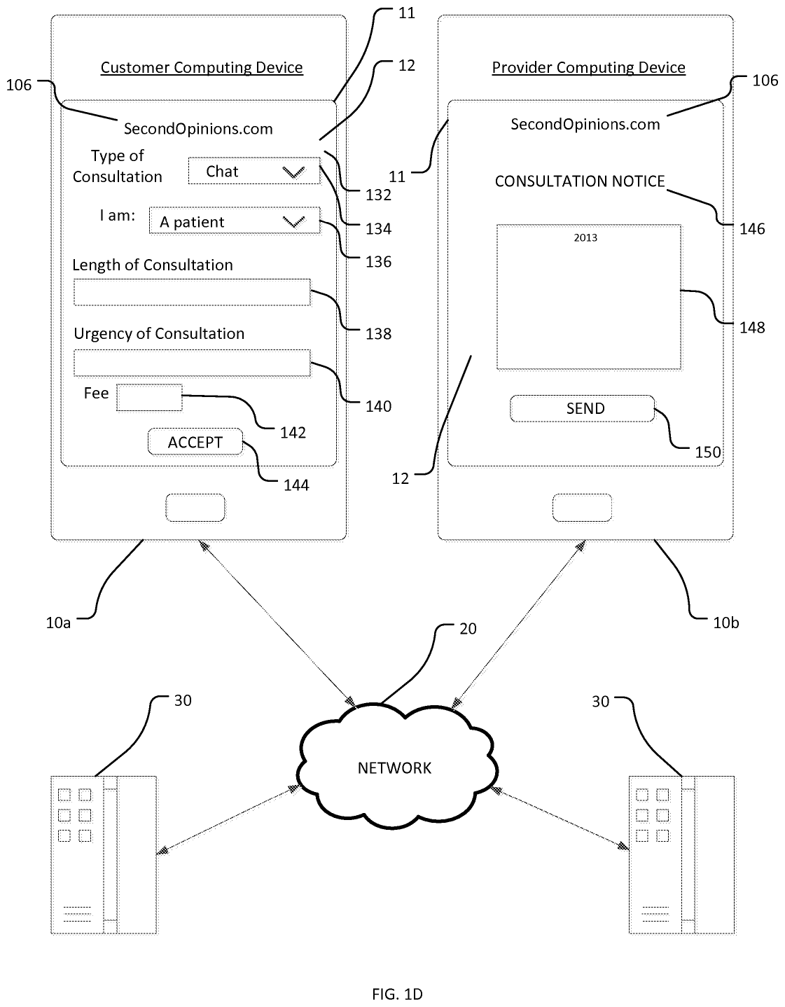

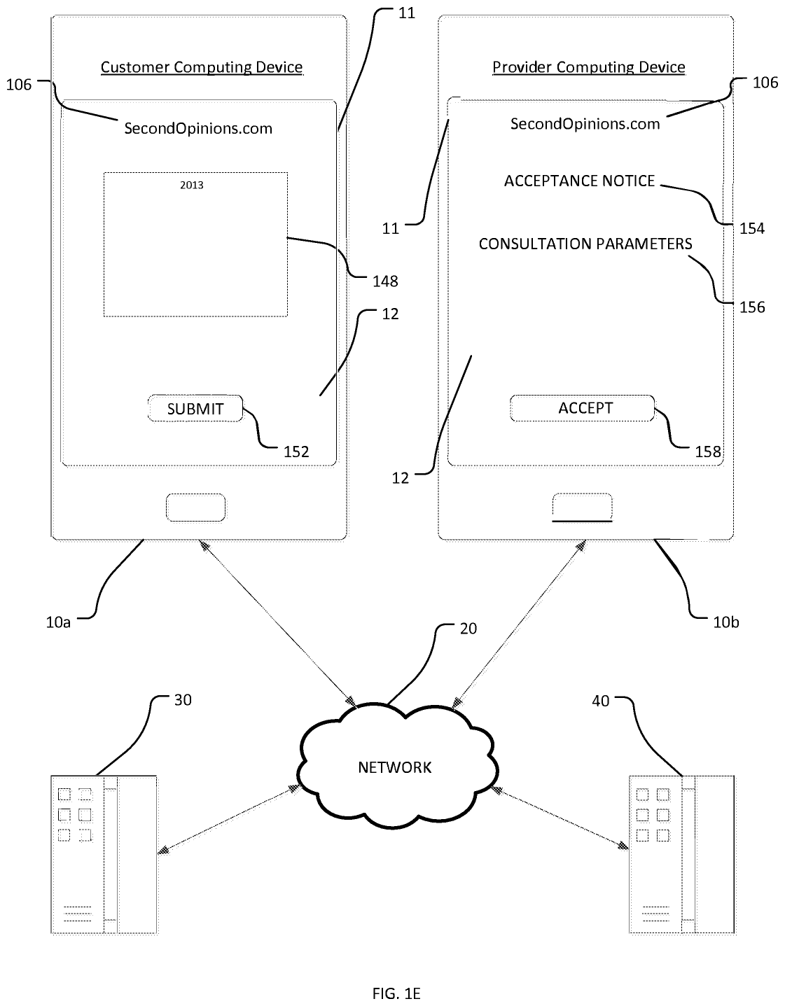

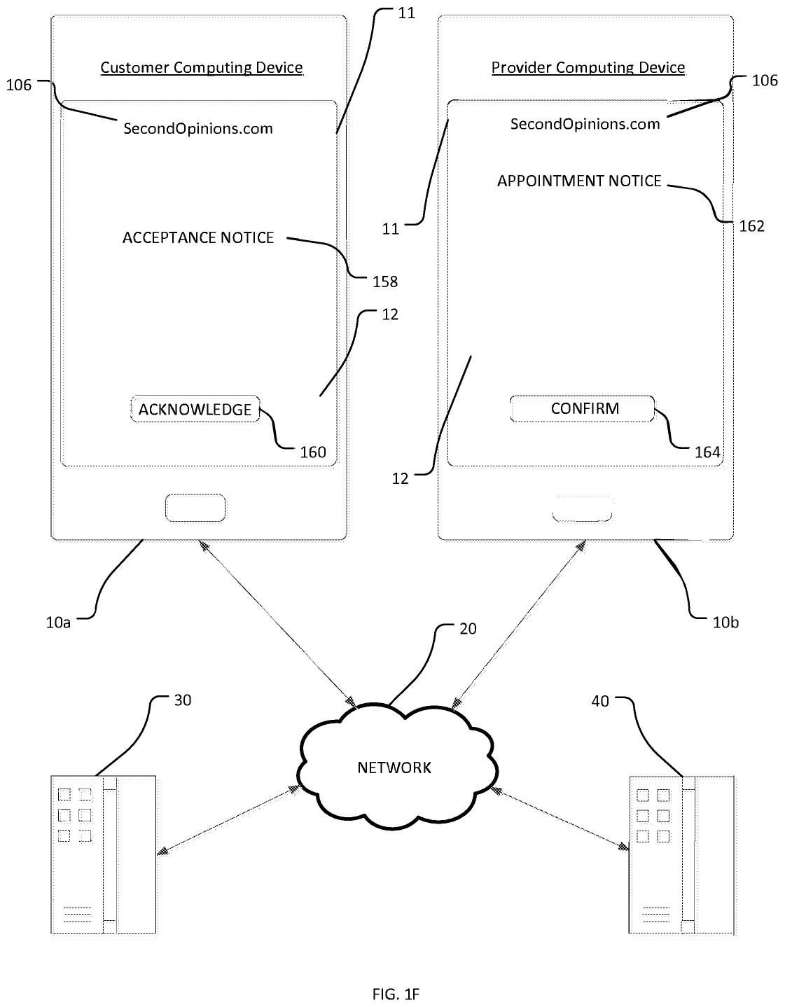

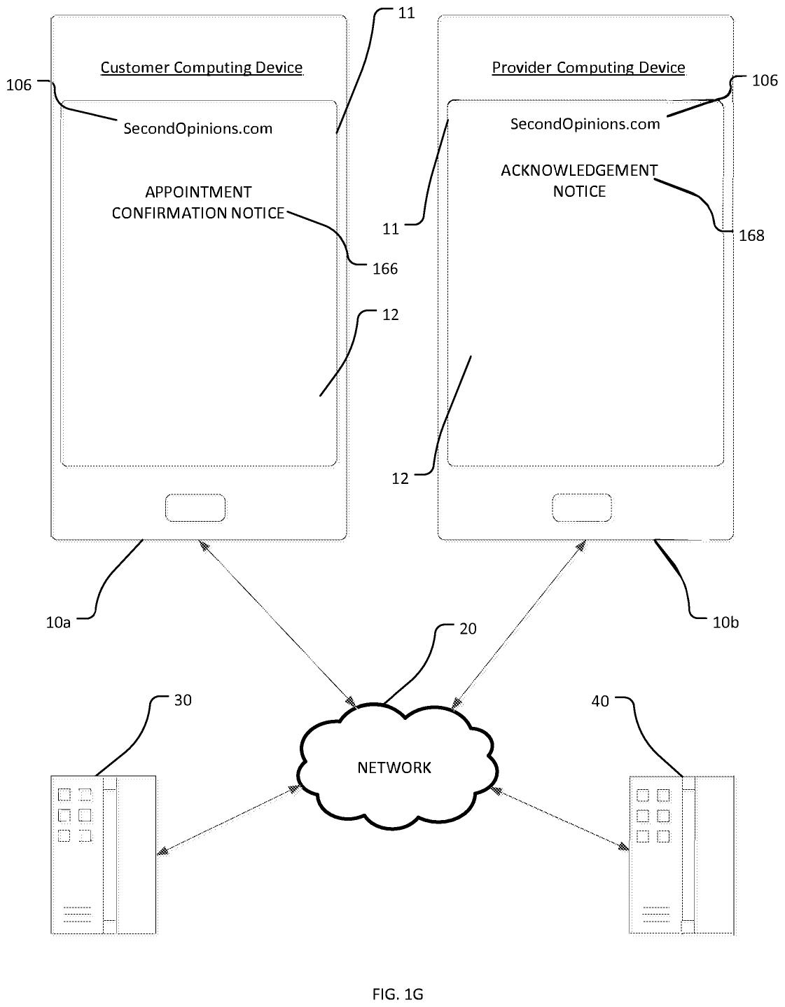

Referring now to FIG. 1A, computing devices 10a and 10b in communication with a brokerage server 30 via a network 20 are illustrated. Computing device 10a can be a computing device used by a consumer while computing device 10b can be a computing device used by an expert. While one brokerage server 30 is illustrated, the term “brokerage server” refers to one or more servers that operate in an individual or distributed manner. Further, as used herein, the term “network” can refer to any communication network including, but not limited to, a wireless network, a cellular network, an intranet, the Internet, or combinations thereof. In the illustrated example, the computing devices 10a and 10b are displaying a graphical user interface (GUI) 12 on a touch screen 11 of the computing devices 10a and 10b. While a touch screen 11 is illustrated, it should be appreciated that other user interfaces can be used to allow the consumer and the expert to interact with their respective computing devices 10a and 10b. For example, in some embodiments of the present disclosure the user interface may employ voice recognition techniques to accept consumer and expert user input.

A third party server 40 is also illustrated. The third party server 40 can be a telemedicine or telehealth server. Operation of the third party server 40 is described in greater detail below.

In one embodiment, the GUI 12 of the computing device 10a allows the consumer to send a request for a second opinion to the brokerage server 30. From a SecondOpinions.com application represented by the icon 106, the GUI 12 can display a pull down menu 100 from which the consumer can select from a plurality of categories identifying the type of second opinion being sought by the consumer. The GUI 12 can also display a field 102 that allows the consumer to enter a description identifying the subject matter of the second opinion being sought. The GUI 12 can further display a button 104 that indicates that the consumer is requesting a second opinion from an expert.

When the consumer has selected the type of second opinion he is looking for and entered a description identifying the subject matter of the second opinion, he can press, or otherwise select, the button 104 to request a list of experts available and qualified to provide an expert second opinion. When the consumer presses the button 104, the computing device 10a can transmit a request to provide a list of experts to the brokerage server 30 via the network 20. The request can include the information entered by the consumer. In some embodiments, the computing device 10a can present additional menus to the consumer, such that the menus prompt the consumer to select characteristics and features of the expert he is seeking.

As illustrated, the GUI 12 of the computing device 10b can display the SecondOpinions.com application icon 106 and a button 108 that upon being selected by the expert connects the expert with the brokerage server 30.

The brokerage server 30 can receive the request from the computing device 10a and can perform a request analysis to specify and formalize the consumer's request and to determine a geographical location of the consumer. In some embodiments, the consumer's request can be analyzed and represented by at least one keyword.

The brokerage server 30 can then query a database for experts that match the consumer's request. In some embodiments, more than one expert can match the consumer's request and a list of experts can be generated by the brokerage sever 30. The brokerage server 30 transmits the list of experts back to the computing device 10a.

Upon receiving the list of experts from the brokerage server 30, the computing device 10a can display the list of experts in the GUI 12, thereby allowing the consumer to select an expert from the list of experts. FIG. 1B illustrates an example of the computing device 10a displaying a list of experts 110. In some embodiments, the list of experts 110 can include experts that are currently online. In other embodiments, the list of experts can include a profile of each expert. The GUI 12 can further display a button 112 that indicates that the consumer is selecting an expert from the list of experts 110.

The brokerage server 30 can send a notice 114 to the computing device 10b that can be displayed by the GUI 12. The notice can notify the expert that his profile has been sent to a consumer. The GUI 12 can further display a button 116 that indicates that the expert acknowledges receipt of the notice 114.

The brokerage server 30 can receive the consumer's selection and transmit the selected expert's profile to the computing device 10a. FIG. 1C illustrates an example of the computing device 10a displaying the expert profile 118. The GUI 12 can further display a button 120 that indicates that the consumer is requesting a consultation with the expert. The GUI 12 can also display a button 122 that indicates that the consumer is selecting an expert store.

The brokerage server 30 can also transmit a notice and a consumer profile to the computing device 10b. The notice can notify the expert that he has been selected by the consumer. The GUI 12 can display the notice 124 and a consumer profile 126. The consumer profile 126 can include the information included in the request as well as other information such as the consumer's preferred method of communicating with the expert. The GUI 12 can further display a button 128 that indicates that the expert acknowledges receipt of the notice 124 and the consumer profile 126. The GUI 12 can also display a button 130 that indicates that the expert is contacting the consumer.

The brokerage server 30 can receive the consumer's consultation request and transmit a fee calculator to the computing device 10a. FIG. 1D illustrates an example of the computing device 10a displaying the fee calculator 132. The fee calculator 132 can include a drop-down menu 134 to indicate a type of consultation preferred by the consumer, a drop-down menu 136 to indicate the nature of consultation requested, a slider 138 to indicate the length of consultation desired by the consumer, a slider 140 to indicate the urgency of the consultation, and a fee indicator 142. In some embodiments, the fee indicator indicates a fee based upon the consultation parameters selected by the consumer in the two drop-down menus 134 and 136 and the sliders 138 and 140. The GUI 12 can also display a button 144 that indicates that the consumer accepts the expert's fee displayed in the fee indicator 142.

The brokerage server 30 can also transmit a notice and an expert calendar to the computing device 10b. The notice can notify the expert that the consumer has requested a consultation. The GUI 12 can display the consultation notice 146 and the expert's calendar 148. In one embodiment, the expert calendar 148 can show the expert's availability for a consultation. The GUI 12 can also display a button 150 that indicates that the expert is sending the expert calendar to the brokerage server 30.

The brokerage server 30 can receive the consumer's acceptance of the expert's fee and the consultation parameters and transmit the expert calendar to the computing device 10a. FIG. 1E illustrates an example of the computing device 10a displaying the expert calendar 148. In some embodiments, the consumer can select a time for a consultation with the expert from the expert's available times. The GUI 12 can display a button 152 that indicates that the consumer is submitting his selection of a date and time for the consultation with the expert.

The brokerage server 30 can also transmit a fee acceptance notice and the consultation parameters to the computing device 10b. The notice can notify the expert that the consumer has accepted the expert's fee. The GUI 12 can display the acceptance notice 154 and the consultation parameters 156. The GUI 12 can also display a button 158 that indicates that the expert is sending his acceptance of the consumer's consultation parameters 156 to the brokerage server 30.

The brokerage server 30 can receive the consumer's date and time submission and transmit an acceptance notice indicating the expert's acceptance of the consumer's consultation parameters 156 to the computing device 10a. FIG. 1F illustrates an example of the computing device 10a displaying an acceptance notice 158. The GUI 12 can display a button 160 that indicates that the consumer acknowledges receipt of the acceptance notice 158.

The brokerage server 30 can also receive the consumer's submission of his selection of a date and time for the consultation with the expert and transmit an appointment notice to the computing device 10b. The appointment notice 162 can include the date and time selected by the consumer. The GUI 12 can display a button 164 that indicates that the expert confirms the appointment at the selected date and time.

The brokerage server 30 can receive the expert's appointment confirmation and transmit an appointment confirmation notice to the computing device 10a. FIG. 1G illustrates an example of the computing device 10a displaying an appointment confirmation notice 166. The brokerage server 30 can also receive the consumer's acknowledgement of receipt of the acceptance notice 158 and transmit an acknowledgement notice to the computing device 10b. The GUI 12 can display the acknowledgement notice 168 on the computing device 10b.

The examples of FIGS. 1A-1G are provided for example only and not intended to be limiting. It should be appreciated that variations of the GUI 12 are contemplated and are within the scope of the disclosure. Furthermore, while mobile computing devices are depicted, the computing devices 10a and 10b can be stationary computing devices as well. Further, in some embodiments of the present disclosure, a second opinion can be generated by the brokerage server in response to the request from the consumer. For example, the brokerage server can employ artificial intelligence to build a database of opinions indexed to particular consumer requests.

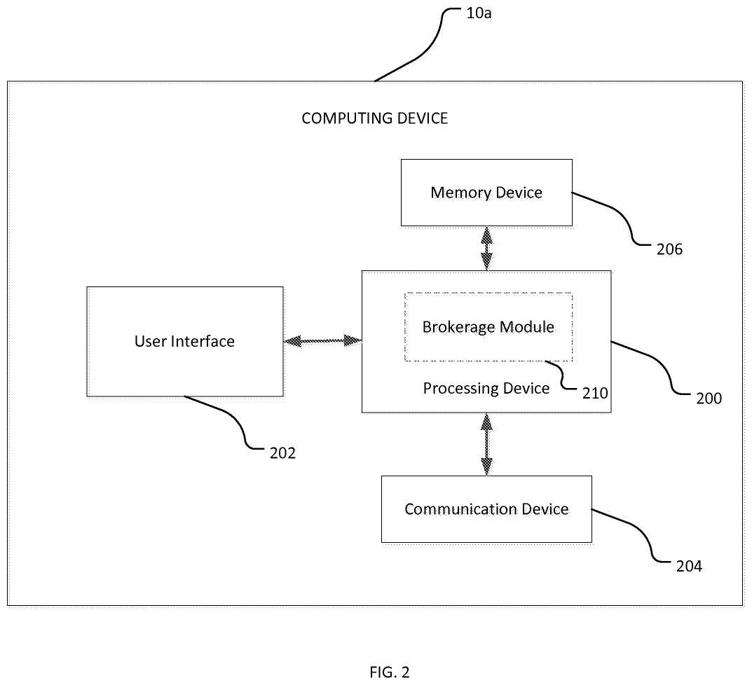

Referring now to FIG. 2, a block diagram illustrating example components of the computing devices 10a and 10b of FIGS. 1A-1G is depicted. Since the computing devices 10a and 10b are identical in all structural respects, the computing device 10a will be described. In the example embodiment, the computing device 10a includes a processing device 200, a user interface 202, a communication device 204, and a memory device 206.

The processing device 200 can include memory, e.g., read only memory (ROM) and random access memory (RAM), storing processor-executable instructions and one or more processors that execute the processor-executable instructions. In embodiments where the processing device 200 includes two or more processors, the processors can operate in a parallel or distributed manner. The processing device 200 can execute the operating system of the computing device 10a. In the illustrative embodiment, the processing device 200 also executes a brokerage module 210, which is described in greater detail below. The brokerage module 210 performs different functions dependent upon whether it is being executed by the computing device 10a or the computing device 10b.

The user interface 202 is a device that allows the consumer to interact with the computing device 10a. While one user interface 202 is shown, the term “user interface” can include, but is not limited to, a touch screen, a physical keyboard, a mouse, a microphone, and/or a speaker. The communication device 204 is a device that allows the computing device 10a to communicate with another device, e.g., the brokerage server 30, via the network 20. The communication device 204 can include one or more wireless transceivers for performing wireless communication and/or one or more communication ports for performing wired communication. The memory device 206 is a device that stores data generated or received by the computing device 10a. The memory device 206 can include, but is not limited to a hard disc drive, an optical disc drive, and/or a flash memory drive.

In some embodiments, the brokerage module 210 is embodied as processor-executable instructions stored in the memory of the processing device 200. The processing device 200 can execute the brokerage module 210. The brokerage module 210 can be preloaded into the operating system of the computing device 10a, can be downloaded from a third party server by a consumer of the computing device 10a, or be generated on the computing device 10a.

The brokerage module 210 of the computing device 10a provides the GUI 12 and receives consumer instructions via consumer input. The brokerage module 210 further receives commands to request an expert second opinion from the brokerage server 30. In response to receiving a list of experts from the brokerage server 30, the brokerage module 210 displays the list of experts in the GUI 12 and receives consumer input indicating a selection of one of the experts. In response to receiving an expert profile from the brokerage server 30, the brokerage module 210 displays the expert profile in the GUI 12 and receives consumer input indicating a request for a consultation with the expert. In response to receiving a fee calculator from the brokerage server 30, the brokerage module 210 displays the fee calculator in the GUI 12 and receives consumer input indicating consultation parameters such as the type of consultation desired (in person, phone, chat, video) and the length and urgency of the consultation. The brokerage module 210 further receives an expert calendar from the brokerage sever 30 and displays the expert calendar in the GUI and receives consumer input indicating a selection of a date and time for the consultation. It should be appreciated that the brokerage module 210 of the computing device 10a can be configured to perform additional functions without departing from the scope of the present disclosure.

The brokerage module 210 of the computing device 10b provides the GUI 12 and receives expert instruction via expert input. The brokerage module 210 further receives commands to acknowledge receipt of a consumer profile from the brokerage server 30. In response to receiving a consultation request from the brokerage server 30, the brokerage module 210 displays an expert calendar in the GUI 12 and receives expert input indicating the expert's intention to send the expert calendar to the brokerage server 30. In response to receiving consultation parameters from the brokerage server 30, the brokerage module 210 displays the consultation parameters in the GUI 12 and receives expert input indicating the expert's acceptance of the consultation parameters. In response to receiving an appointment notice from the brokerage server 30, the brokerage module 210 displays the appointment notice in the GUI 12 and receives expert input indicating confirmation of the appointment. It should be appreciated that the brokerage module 210 of the computing device 10b can be configured to perform additional functions without departing from the scope of the present disclosure.

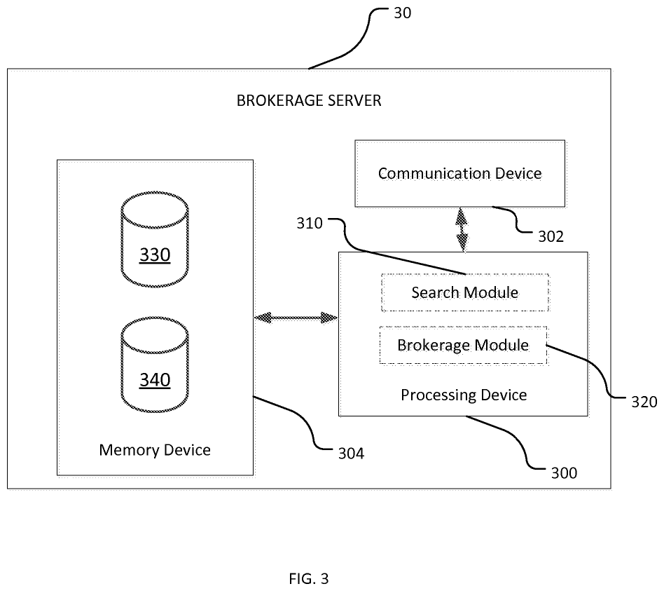

Referring now to FIG. 3, a block diagram illustrating an exemplary brokerage server 30 is depicted. In an exemplary embodiment, the brokerage server includes a processing device 300, a communication device 302, and a memory device 304.

The processing device 300 can include memory, e.g., read only memory (ROM) and random access memory (RAM), storing processor-executable instructions and one or more processors that execute the processor-executable instructions. In embodiments where the processing device 300 includes two or more processors, the processors can operate in a parallel or distributed manner. In the illustrative embodiment, the processing device 300 executes a search module 310 and a brokerage module 320, which are described in greater detail below.

The communication device 302 is a device that allows the brokerage server 30 to communicate with another device, e.g., the computing devices 10a and 10b, via the network 20. The communication device 302 can include one or more wireless transceivers for performing wireless communication and/or one or more communication ports for performing wired communication.

The memory device 304 is a device that stores data generated or received by the brokerage server 20. The memory device 304 can include, but is not limited to a hard disc drive, an optical disc drive, and/or a flash memory drive. The memory device 304 is accessible to the processing device 300. A participant database 330 and a knowledge database 340, or similar structures, can be stored in the memory device 304.

The participant database 330 stores information related to registered consumers and experts. Information stored in the participant database 330 can include consumer data such as name, address, and date of birth as well as queries made by the consumer. Information stored in the participant database 330 can also include expert data such as name, address, and date of birth, as well as areas of expertise and qualifications of the expert.

The knowledge database 340 stores information related to consumer queries. The knowledge database 340 can include text, audio and video files and other relevant files that can be transmitted to the consumer computing device 10a by the brokerage server 30. The knowledge database 340 can include artificial intelligence functionality that provides for indexing of records to particular consumer queries.

The search module 310 receives commands from the computing device 10a. The search module 310 receives a request for an expert second opinion from the computing device 10a. As described, the request can include a description of the consumer and the subject matter of the second opinion being sought by the consumer. The search module 310 performs a request analysis to specify and formalize the consumer's request and to determine a geographical location of the consumer. The consumer's location can be determined using location-based technologies such as mobile GPS, HTMLS, and AJAX. In some embodiments, the consumer's request can be analyzed and represented by at least one keyword.

The search module 310 next queries the participant database 320 and determines if there is a match between the consumer's request and an available expert. The search module 310 can send a list of experts matching the consumer's request to the communication device 302 that in turn transmits the list of experts to the computing device 10a. In other embodiments of the present disclosure, the search module 310 searches the knowledge database 340 and determines if there are any records related to the consumer's request. In some embodiments of the present disclosure, the search module 310 sends any records related to the consumer's request to the communication device 302 that in turn transmits the records to the computing device 10a.

The brokerage module 320 receives commands from the computing devices 10a and 10b. In response to receiving a consumer's selection of an expert from the list of experts, the brokerage module 310 notifies the computing device 10b of the selected expert, provides the consumer's profile to the computing device 10b, and provides the expert's profile to the computing device 10a. In some embodiments, the consumer and expert profiles can be stored in the participant database 320.

The brokerage module 320 receives a consultation notice from the computing device 10a and sends the computing device 10a an expert fee calculator. In some embodiments, the expert fee calculator can include consumer-selectable fields indicating consultation parameters such as the type of consultation sought and the length and urgency of the consultation. The consultation parameters can be used to determine the expert's fee. In response to receiving a consumer's acceptance of the fee determined by the expert fee calculator, the brokerage module 320 sends the acceptance notice and the consultation parameters to the computing device 10b and the expert's calendar to the computing device 10a in case the expert accepts the consumer's consultation parameters. In response to receiving a consumer's submission of a selected date and time for the consultation with the selected expert, the brokerage module 320 sends an appointment notice to the computing device 10b.

In some embodiments of the present disclosure, the consultation between the consumer and the expert can be mediated by the brokerage module 320. For example, the brokerage module 320 can facilitate an online chat between the consumer and the expert. In other embodiments, the consultation between the consumer and the expert can be mediated by the brokerage module 320 and include the facilities of the third party server 40. For example, in the case where one medical professional is seeking a second opinion from another medical professional, the third party server 40 can be a telemedicine server. In the case where the consumer is a patient seeking a medical second opinion, the third party server 40 can be a telehealth server.

It is appreciated that the foregoing example of the brokerage server 30 is not intended to be limiting. Variations of the exemplary brokerage server 30 are contemplated and within the scope of the invention. For example, the memory device 304 may store a look-up table or a hash table that relates consumer requests to potential experts. In another example, the brokerage server 30 can utilize artificial intelligence techniques to associate keywords with each record in the participant database 330 to increase the robustness of the consumer/expert matching process.

The brokerage server 30 can also include modules that provide administrative and security functions. For example, the processing device 300 can execute a registration module to provide consumers and experts with an interface for entering registration information such as name, address, date of birth, medical conditions, consultation preferences, and qualifications. Consumer and expert relations modules can be executed by the processing device 300 to provide consumers and experts with an interface for managing their accounts. In other exemplary embodiments, the processing device 300 can execute a coupon module that provides registered experts with the ability to post promotions and coupons. In other exemplary embodiments, the processing device 300 can execute a review module that provides registered consumers and experts with the ability to post reviews of a consultation.

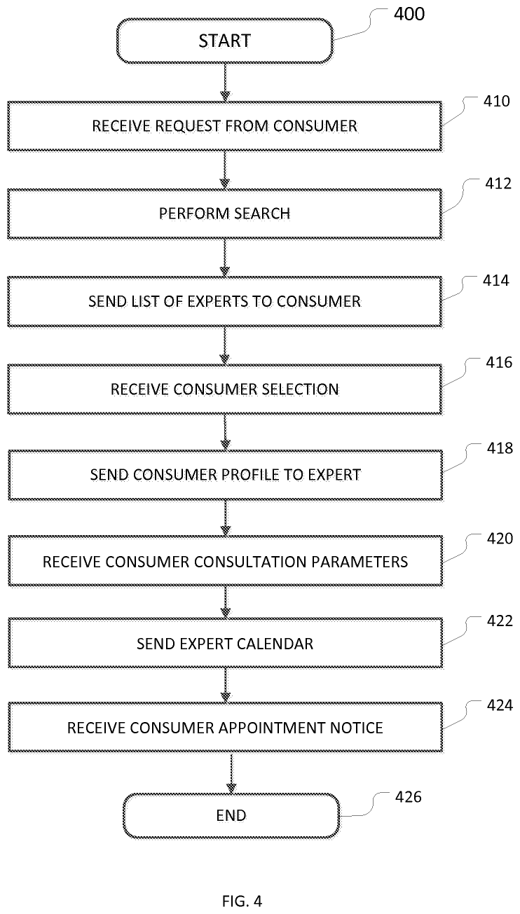

Referring now to FIG. 4, an example method 400 for automating the provision of a second opinion is illustrated. In some embodiments the brokerage server 30 (FIG. 3) executes the method 400. The flowchart and block diagrams in the flow diagrams illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

At operation 410, the search module 310 receives the request from the consumer's computing device 10a. As discussed above, the GUI 12 of the consumer's computing device 10a allows the consumer to enter the request. At operation 412, the search module 310 conducts a search and determines if there is a match between the consumer's request and an expert. At operation 414, the search module 310 provides the list of experts to the consumer's computing device 10a. In some embodiments, the list of experts can include an expert profile for each expert in the list.

At operation 416, the brokerage module 320 receives a consumer selection indicating the consumer's desire to consult with the selected expert. At operation 418, the brokerage module 320 sends the consumer's profile to the expert's computing device 10b. At operation 420, the brokerage module 320 receives consultation parameters from the consumer's computing device 10a. As previously discussed, the consultation parameters can include an expert fee. At operation 422, the brokerage module 320 sends the consumer's computing device 10a the expert's calendar. The experts calendar can show the customer the dates and times when the expert is available for a consultation. At operation 424, the brokerage module 320 receives the consumer's appointment notice indicating the consumer's selection of the date and time for the consultation.

The method 400 of FIG. 4 is provided for example and is not intended to be limiting. It should be appreciated that the method 400 can include additional operations and some operations may be varied without departing from the scope of the disclosure.

In one embodiment, the present disclosure utilizes blockchain technology to govern disputes, govern electronic payments, and establish communication directly between parties without third-party intermediaries. Blockchains are decentralized, peer-to-peer networked databases that can store and authenticate a ledger of assets and asset transactions. Blockchain technology allows transactions to occur without a third-party intermediary. Blockchain technology uses smart contracts to resolve disputes. Smart contracts are computer programs designed to act as self-executing contracts that implement aspects of a transaction where the terms of the agreement between buyer and seller are directly written into lines of software code. The code and the agreements exist across a distributed, decentralized blockchain network.

The present disclosure allows for transactions to take place across a blockchain database. For example, any electronic payment that is sent by consumer following, for example, a brokered consultation between the consumer and an expert, can be governed by blockchain technology, specifically using a smart contracts concept, which allows for instantaneous payment.

Further, the present disclosure allows for all expert second opinions to be stored in a blockchain database.

In one embodiment, processing device 300 automatically selects one or more experts from a pool of experts to render a second opinion. This selection can be based on, for example, expert characteristics and expertise.

In another embodiment, artificial intelligence (“AI”) algorithms are used to help experts provide second opinions faster and more accurately.

The above description of illustrated examples of the present invention, including what is described in the Abstract, are not intended to be exhaustive or to be limitation to the precise forms disclosed. While specific embodiments of, and examples for, the invention are described herein for illustrative purposes, various equivalent modifications are possible without departing from the broader spirit and scope of the present invention. Indeed, it is appreciated that the specific example voltages, currents, frequencies, power range values, times, etc., are provided for explanation purposes and that other values may also be employed in other embodiments and examples in accordance with the teachings of the present invention.

We claim:

1. A computer-implemented method comprising the steps of:
receiving, at a processing device of a brokerage server, a request from a first remote computing device, the request being indicative of a request by a consumer for a second opinion from an expert;
determining, at the processing device, a list of one or more experts who match the request;
providing, from the processing device, the list of the one or more experts to the first remote computing device; and
brokering, at the processing device, a consultation between the consumer and the expert.

2. The computer-implemented method of claim 1, further comprising analyzing the request and representing the request as at least one keyword.

3. The computer-implemented method of claim 1, further comprising determining a geographical location of the first remote computing device.

4. The computer-implemented method of claim 1, wherein brokering the consultation between the consumer and the expert further comprises:
receiving, at the processing device, a selection from the first remote computing device, the selection being indicative of a consumer selection of an expert from the list; and
providing, from the processing device, a consumer profile to a second remote computing device, the consumer profile providing the expert with information related to the consumer's request for the second opinion.

5. The computer-implemented method of claim 4, wherein brokering the consultation between the consumer and the expert further comprises:
receiving, at the processing device, consumer consultation parameters from the first remote computing device;
providing, from the processing device, the consumer consultation parameters to the second remote computing device; and
receiving, at the processing device, an acceptance from the second remote computing device, the acceptance being indicative of acceptance by the expert of the consumer consultation parameters.

6. The computer-implemented method of claim 5, wherein brokering the consultation between the consumer and the expert further comprises:
providing, from the processing device, an expert calendar to the first remote computing device;
receiving, at the processing device, an appointment notice from the first remote computing device, the appointment notice being indicative of a consumer selection of an appointment date and time with the expert;
providing, from the processing device, the appointment notice to the second remote computing device.

7. The computer-implemented method of claim 6, wherein brokering the consultation between the consumer and the expert further comprises:
receiving, at the processing device, a confirmation from the second remote computing device, the confirmation being indicative of the expert's confirmation of the consumer-selected appointment date and time; and
providing, from the processing device, an appointment confirmation notice to the first remote computing device.

8. A brokerage server having a processing device, the brokerage server comprising:
a search module configured to:
(a) receive a request from a first remote computing device, the request being indicative of a request by a consumer for a second opinion from an expert;
(b) determine a list of one or more experts that match the request; and
(c) provide the list of one or more expert to the first remote computing device; and
a brokerage module configured to:
(a) broker a consultation between the consumer and the expert.

9. The brokerage server of claim 8, wherein the brokerage module is further configured to receive a selection from the first remote computing device, the selection being indicative of a consumer selection of an expert from the list, and to provide a consumer profile to a second remote computing device, the consumer profile providing the expert with information related to the consumer's request for the second opinion.

10. The brokerage server of claim 9, wherein the brokerage module is further configured to receive consumer consultation parameters from the first remote computing device, provide the consumer consultation parameters to the second remote computing device, and receive an acceptance from the second remote computing device, the acceptance being indicative of acceptance by the expert of the consumer consultation parameters.

11. The brokerage server of claim 10, wherein the brokerage module is further configured to provide an expert calendar to the first remote computing device, receive an appointment notice from the first remote computing device, the appointment notice being indicative of a consumer selection of an appointment date and time with the expert, and provide the appointment notice to the second remote computing device.

12. The brokerage server of claim 11, wherein the brokerage module is further configured to receive a confirmation from the second remote computing device, the confirmation being indicative of the expert's confirmation of the consumer-selected appointment date and time, and provide an appointment confirmation notice to the first remote computing device.
